SEQUENCE LISTING (6 sequences)

SEQ ID NO: 1 - DNA, 25 bp, Artificial Sequence
  synthetic
gcgagtttttttttttttttctcgc

SEQ ID NO: 2 - DNA, 20 bp, Artificial Sequence
  synthetic
aaaaaaaaaaaaaaactcgc

SEQ ID NO: 3 - DNA, 21 bp, Artificial Sequence
  synthetic
gaaaaaaaaaaaaaaactcgc

SEQ ID NO: 4 - DNA, 21 bp, Artificial Sequence
  synthetic
gaaaaaaacaaaaaaactcgc

SEQ ID NO: 5 - DNA, 70 bp, Artificial Sequence
  synthetic
ctacctacagtaccaagcttnnnnnnnnnnnnnnnnnnnnnnnnnnnnnnttactcgagggatcctagtc

SEQ ID NO: 6 - DNA, 25 bp, Artificial Sequence
  synthetic
gcgagtttttttttttttttctcgn

(19) United States
(12) Patent Application Publication
(10) Pub. No.: US 2004/0002089 A1
(43) Pub. Date: Jan. 1, 2004

(54) Methods employing fluorescence quenching by metal surfaces

(76) Inventors: Benoit Dubertret, New York, NY (US); Michel Calame, Riehen (CH); Albert Libchaber, New York, NY (US)

Correspondence Address: KLAUBER & JACKSON, 411 HACKENSACK AVENUE, HACKENSACK, NJ, 07601

(21) Appl. No.: 10/374,686

(22) Filed: Feb. 26, 2003

Related U.S. Application Data
(63) Continuation of application No. PCT/US01/41941, filed on Aug. 29, 2001.
(60) Provisional application No. 60/228,728, filed on Aug. 29, 2000.
(60) Provisional application No. 60/280,350, filed on Mar. 30, 2001.

(51) Int. Cl. C12Q 1/68; G01N 33/553

(52) U.S. Cl. 435/6; 436/525

(57) ABSTRACT

The invention is broadly directed to methods for sensitively detecting proximity changes in systems that utilizes an interacting fluorophore and quencher. In such methods, a metal surface is used as the quencher. The metal surface may be a particle or film, such as nanoparticles or a coating, respectively. Such systems provide an increase in sensitivity over previously-described quenchers, offering a signal-to-noise ratio of up to several orders of magnitude. Examples of such systems in which proximity changes are usefully detected include conformational changes in biomolecules resulting from their interaction with their binding partners or ligands. Such biomolecules may be, for example, nucleic acids, proteins, peptides, polysaccharides, or other polymeric, naturally occurring or synthetic molecules. These include, by way of non-limiting example, molecular beacons, which detect particular polynucleotide sequences; antibody-antigen interactions, and conformational changes in proteins upon binding to a ligand or substrate.

### CROSS-REFERENCE TO RELATED APPLICATIONS

This application is a Continuation of International Application PCT/US01/41941, filed Aug. 29, 2001, which in turn, claims benefit of priority under 35 U.S.C. 119(e) to Provisional Application Serial No. 60/228,728, filed Aug. 29, 2000 and to Provisional Application Serial No. 60/280,350, filed Mar. 30, 2001. Applicants claim the benefit of 35 U.S.C. 120 as to said International Application, and all of said applications are incorporated by reference herein in their entireties.

### FIELD OF THE INVENTION

This invention relates to the use of metal surface quenchers such as particles or films for high sensitivity applications in, for example, detection and diagnostic systems.

### BACKGROUND OF THE INVENTION

Hybrid materials composed of biomolecules, such as proteins or polynucleotides, and non-biological inorganic objects (for example tiny particles of insulators, semi-conductors and metals) have recently been assembled (Mirkin C. A. et al. (1996) Nature 382:607-609; Alivisatos A. P. et al. (1996) Nature 382: 609-611; Mitchell G. P. et al. (1999) J. Am. Chem. Soc. 121: 8122-8123; Whaley S. R. et al. (2000) Nature 405: 665-668). The highly specific recognition properties of biomolecules combined with the unique optical properties of the inorganic nanoparticles make these composite materials attractive for use in the fields of biodiagnostics (non-photobleaching immunolabels) (Schultz S. et al. (2000) Proc. Natl. Acad. Sci. 97: 996-1001), sensitive probes for polynucleotides detection (Elghanian R. et al. (1997) Science 277: 1078-1081; Storhoff J. J. (1998) J. Am. Chem. Soc. 120: 1959-1964) and nanotechnologies (Whaley S. R. et al. (2000) Nature 405: 665-668). For example, gold nanocrystals may self-assemble (Loweth C. J. et al. (1999) Angew Chem Int Edit 38: 1808-1812; Niemeyer C. M. et al. (1998) Angew Chem Int Edit 37: 2265-2268), with good spatial control of the inorganic components, broadening the scope for “bottom-up” fabrication. Although some fundamentally interesting properties of hybrid materials have already been discovered (Schultz S. et al. (2000) Proc. Natl. Acad. Sci. 97: 996-1001; Storhoff J. J. et al. (2000) J. Am. Chem. Soc. 122: 4640-4650), their use as functional materials is still very limited.

The unique thermodynamics and specificity of molecular beacons have been studied (Bonnet G. et al. (1999) Proc. Natl. Acad. Sci. 96: 6171-6176; Bonnet G. et al. (1998) Proc. Natl. Acad. Sci. 95: 8602-8606) and the probe has two main advantages. It has an excellent sensitivity to the detection of one mismatch in a sequence of nucleic acid, and it allows the direct detection of unlabeled oligonucleotides. Although these unique properties yielded new results in areas such as genomics (Marras S. A. E. et al. (1999) Genet. Anal. Biomol. E. 14: 151-156), DNA chips (Steemers F. J. et al. (2000) Nat. Biotechnol. 18: 91-94) or quantitative PCR (Tyagi S. et al. (1997) Nature Biotechnol. 16: 49-53; Vet J. A. M. et al. (1999) Proc. Natl. Acad. Sci. 96: 6394-6399), one great challenge remains to improve the signal to noise: the efficiency of the quenching of the fluorescence.

The organic quencher used at present, the 4-([4′-(dimethyl-amino)phenyl]azo)benzoic acid (DABCYL) quenches at most 99% of the fluorescence of the dye placed in its proximity. DABCYL optimally quenches fluorescein, but its quenching efficiency decreases for dyes emitting at longer wavelengths (DABCYL absorbs at best 20% of the fluorescence of Cy5 [indodicarbocyanine] whose maximum of emission is at 670 nm). A better quencher would greatly increase the sensitivity and the possible application of the molecular beacons.

It is towards improving the sensitivity of various hybrid molecules employing fluorophore-quencher interactions that the present invention is directed.

The citation of any reference herein should not be construed as an admission that such reference is available as “Prior Art” to the instant application.

### SUMMARY OF THE INVENTION

The invention is broadly directed to methods for sensitively detecting proximity changes in systems that utilizes an interacting fluorophore and quencher. In such methods, a metal surface is used as the quencher. The metal surface may be a particle or film, such as nanoparticles or a coating, respectively. The invention is also broadly directed to detectable compositions comprising a fluorophore, a metal surface, and a molecule whose conformational changes in response to interaction with another moiety or condition result in a change in fluorescence of the composition. Such compositions are hybrid molecules comprising the aforementioned three components; in the instance where the metal surface is a film and not a particle, the hybrid molecule may be tethered to the metal surface, and still exhibit its conformational-change-detecting properties.

Such systems provide an increase in sensitivity over previously-described quenchers, offering a signal-to-noise ratio of up to several orders of magnitude. Examples of such systems in which proximity changes are usefully detected include conformational changes in biomolecules resulting from their interaction with their binding partners or ligands. Such biomolecules may be, for example, nucleic acids, proteins, peptides, polysaccharides, or other polymeric, naturally-occurring or synthetic molecules. These include, by way of non-limiting example, molecular beacons, which detect particular polynucleotide sequences; antibody-antigen interactions, and conformational changes in proteins upon binding to a ligand or substrate.

Preferred metal surfaces include metal particles and metal films. Preferred metal particles or clusters are nanoparticles, more preferably gold nanoparticles, silver nanoparticles and palladium nanoparticles, or combinations thereof. Such clusters or nanoparticles may comprise from 3 to up to about 10⁷ atoms. Other metals include but are not limited to lithium, sodium, copper, aluminum, magnesium, barium, potassium, rubidium, and cesium. The particles may comprise mixtures or combinations of metal atoms. Gold nanoparticles are most preferred. The gold nanoparticle has a diameter greater than 0.8 nm, comprising more than 11 gold atoms, and preferably about 1.4 nm, comprising about 50 to 70 gold atoms. The metal nanoparticle or film may be derivatized to covalently or otherwise bind to and form the hybrid molecule. Coating of the metal surface with one or more polymers to provide a hydrophobic, hydrophilic or otherwise charged surface, or providing functional groups or groups that can subsequently be modified to provide functional groups or those that can associate with the desired biomolecule(s), may be carried out to facilitate the preparation of the associated quencher(s) with the modified biomolecule(s). Such modification may also increase the water solubility of metal particles. The metal nanoparticle and fluorophore may be provided on separate, interacting hybrid molecules. Metal films include coatings or films comprising one or combinations of the aforementioned metals.

Metal surfaces include metal films, and such films can be smooth, such as when gold is evaporated on a smooth surface, or they can be rough, such as when gold colloids are adsorbed and partially melted on an evaporated gold surface. The foregoing are merely examples of various types of metal surfaces useful as quenchers for the applications described herein, and variations therein are fully embraced by the present invention.

The fluorophore may be any fluorophore whose light output may be quenched by a metal surface. Non-limiting examples include a luminescent metal, a luminescent semiconductor, a fluorescent organic dye, a fluorescent protein or a fluorescent peptide. A non-limiting example of a luminescent semiconductor is a quantum dot. Non-limiting examples of a fluorescent organic dye include fluorescein and its derivatives, rhodamine and its derivatives, Texas Red, Cy5, acridine orange, 2,7-dichlorofluorescein, eosin, rose bengal, 1,2-dihydroxyanthraquinone, 1,4-dihydroxyanthraquinone, 1,8-dihydroxyanthraquinone, 1,3,8-trihydroxy-6-ethylanthraquinone, 1,2,5,8-tetrahydroxyanthraquinone, 1-aminonaphthalene, and 2-aminonaphthalene. Fluorescent derivatives of any of the foregoing are further examples of suitable fluorophores. The foregoing are merely examples and are not intended to be exhaustive.

The fluorescent protein may be, for example, green fluorescent protein. Fluorescent peptides are also embraced herein. The invention is not so limiting as to particular fluorophores but to pairs comprising a fluorophore and a metal surface such that proximity therebetween results in quenching of the fluorophore.

As noted above the increased sensitivity may be provided as an increased ratio of signal to noise; preferably, the ratio of signal to noise is increased at least two-fold, or as much as up to ten-fold, a hundred-fold, or as high as a thousand-fold or more as compared to quenchers that are not metal surfaces.

In one aspect, compositions of the invention comprise three components: a metal surface, such as a metal particle or metal film as mentioned above, a fluorophore such as mentioned above, and a molecule whose conformational changes are desirably detected. Thus, a hybrid molecule may comprise 1) a metal particle or metal film, such as a metal nanoparticles, preferably gold nanoparticles, silver nanoparticles and palladium nanoparticles, or combinations thereof; or those of lithium, sodium, copper, aluminum, magnesium, barium, potassium, rubidium, and cesium, mixtures or combinations; 2) a molecule whose conformational changes are desirably detected, such as but not limited to a nucleic acid, protein, peptide, polysaccharide, glycoprotein, glycolipid, or other polymeric, naturally-occurring or synthetic molecule, such as a molecular beacon, antibody, lectin, receptor, enzyme substrate, and the like, and 3) a fluorophore, such as luminescent metal, a luminescent semiconductor, a fluorescent organic dye, a fluorescent protein or a fluorescent peptide, including but not limited to quantum dots, fluorescein and its derivatives, rhodamine and its derivatives, Texas Red, Cy5, acridine orange, 2,7-dichlorofluorescein, eosin, rose bengal, 1,2-dihydroxyanthraquinone, 1,4-dihydroxyanthraquinone, 1,8-dihydroxyanthraquinone, 1,3,8-trihydroxy-6-ethylanthraquinone, 1,2,5,8-tetrahydroxyanthraquinone, 1-aminonaphthalene, and 2-aminonaphthalene; green fluorescent protein, fluorescent peptides, to name a few.

In a preferred embodiment, the system of the invention is a molecular beacon in which the quenching moiety is a metal nanoparticle. Preferably, the metal nanoparticle is a gold nanoparticle, with a diameter greater than 0.8 nm and having more than about 11 gold atoms; preferably about 1.4 nm with about 50-70 gold atoms. The metal nanoparticle may be derivatized to covalently bind to form the molecular beacon. The fluorophore may be a luminescent semiconductor, a fluorescent organic dye, a fluorescent protein or a fluorescent peptide; non-limiting examples are as described hereinabove.

In another embodiment, fluorescently-labeled nucleic acid probes capable of specifically hybridizing with a particular sequence may be affixed to a metal surface. In the absence of interaction between the probe and its binding partner, the probe assumes a particular conformation and distance between the fluorophore and the metal surface, providing a certain extent of quenching. Upon interaction of the probe with its binding partner, the conformation and thus level of quenching and fluorescence changes, the extent proportional to the amount of binding partner present. Similar detection of interactions between any biomolecule and its binding partner which results in a detectable conformational change using fluorophore-metal. surface-quencher interactions may also be identified and quantitated by corresponding methods. In another embodiment, a variety of biomolecules capable of detecting different types of analytes may be used simultaneously in a metal particle or film system, such as any combination of nucleic acid probes, antibodies, receptors, and other specific binding partners for particular analytes. The conformational change of each at its particular fluorescence wavelength can be individually discriminated, permitting a wide range of structurally dissimilar analytes to be concurrently measured in vitro using a sample, or in situ, using an indwelling probe or biosensor, as described above.

In a further embodiment, a method is provided for increasing the signal-to-noise ratio in a hybrid fluorophore-quencher molecule in which the quencher is DABCYL, the increased signal-to-noise ratio provided by substituting for DABCYL a metal nanoparticle. Non-limiting examples of various applications for such hybrid molecules is as described above, as well as features of the metal nanoparticles.

These and other aspects of the present invention will be better appreciated by reference to the following drawings and Detailed Description.

### BRIEF DESCRIPTION OF THE DRAWINGS

FIGS. 1A-B show the structure of a gold-nanoparticle-quenched molecular beacon in A) schematic form, and B) in chemical structural form.

FIGS. 2A-B depict the results of gel electrophoresis of organic fluorophore-DNA-gold nanoparticle and organic fluorophore-DNA complexes.

FIGS. 3A to 3D depict the efficiency of the quenching of gold nanoparticles. The emission spectrum of hairpin DNA coupled to gold and to a) fluorescein, b) rhodamine 6G, c) Texas Red, and d) Cy5.

FIGS. 4A to 4D show the evolution of the fluorescence of a solution containing A): 4.2 nM of gold nanoparticles-DNA-Rhodamine 6G conjugate and 0.6 microM of gold; B)10 nM of molecular beacon, as the target concentration varies from 67 pM to 13 microM.

FIGS. 5A-B show the result of the attachment of organic fluorophore-DNA to a gold surface and interaction with a complementary DNA target.

### DETAILED DESCRIPTION OF THE INVENTION

The following terms are used herein:

A “molecular beacon” is a nucleic acid probe that recognizes and reports the presence of a specific nucleic acid sequence, and is capable of discriminating a single nucleotide difference, for example, a single-nucleotide polymorphism, in the presence of a large excess of wild-type (most common) polynucleotide. The probe is a hairpin-shaped sequence with a central stretch of nucleotides complementary to the target sequence, and termini comprising short mutually complementary sequences. One terminus is covalently bound to a fluorophore and the other to a quenching moiety. When in their native state with hybridized termini, the proximity of the fluorophore and the quencher is such that little or no fluorescence is detectable. Upon hybridization of the central complementary stretch to the target sequence, the hairpin undergoes a spontaneous fluorogenic conformational change. See, for example, U.S. Pat. No. 5,925,517.

A “metal cluster” or “metal nanoparticle” is a particle composed of from 3 to up to about 10⁷ (10 million) metal atoms. Non-limiting examples include alkali metals, alkaline earth metals, noble metals and transition metals, such as, by way of example, gold, silver, palladium, lithium, sodium, copper, aluminum, magnesium, barium, potassium, rubidium, and cesium. A “metal surface” refers to both metal particles, clusters, nanoparticles and the like, as well as to metal films, coatings, and related forms of materials. A metal surface may be smooth or rough.

A “fluorophore” is a material that absorbs light at one wavelength and emits light at another, longer wavelength. Non-limiting examples of fluorophores include metals such as luminescent semiconductor quantum dots (referred to herein as luminescent semiconductor or “quantum dots”; see, for example, Chan et al., 1998, science 281:2016-2018; Bruchez et al., 1998, Science 281:2013-2015; Murray et al., 1993, J., Amer. Chem. Soc. 115:8706-8715; Lakowicz et al., 1999, J. Phys. Chem. B 103:7613-7620); organic dyes such as fluorescein, rhodamine, Texas Red, Cy5, acridine orange, 2,7-dichlorofluorescein, eosin, rose bengal, 1,2-dihydroxyanthraquinone, 1,4-dihydroxyanthraquinone, 1,8-dihydroxyanthraquinone, 1,3,8-trihydroxy-6-ethylanthraquinone, 1,2,5,8-tetrahydroxyanthraquinone, 1-aminonaphthalene, and 2-aminonaphthalene; and fluorescent proteins and peptides such as green fluorescent protein (GFP), fluorescent peptides, and their derivatives (see, for example, Tsien, R. Y. (1998) Annu. Rev. Biochem. 67, 509-544; Miyawaki, A., Liopis, J., Heim, R., McCaffery, J. M., Adams, J. A., Ikura, M. & Tsien, R. Y. (1997) Nature 388, 882-886).

A “quencher” is a material with the ability to absorb visible, infrared or ultraviolet light, particularly the light emitted by a fluorophore as described herein, when located at a certain proximity to the fluorophore.

A “hybrid material” refers to a compound or composition which comprises at least one fluorophore and at least one quencher, the interaction therebetween used for the detection, measurement, or participation of the hybrid material in an event. Typically, the event, such as binding of the hybrid material to a specific ligand, results in a conformational change in the hybrid material, the change being detectable by the change in proximity of the fluorophore to the quencher and the extent of emission of light from the hybrid molecule after exposure to the excitation wavelength of the fluorophore. By way of a non-limiting example, the conformation of and/or conformational changes in a polymeric biomolecule such as a protein, polysaccharide, or nucleic acid may be monitored by linking a fluorophore to at least one location on the biomolecule, and at least one quencher to another location. Fluorescence generated by exposing the fluorophore to an excitation wavelength of light may be modulated by changes in the proximity of the fluorophore and the quencher. As the fluorophore and quencher approach one another, quenching increases; as they move apart, quenching is reduced. Thus, changes in conformation of the biomolecule may be measured by changes in fluorescence. This detectable change in proximity and/or conformation is or may be used in various useful applications such as but not limited to medical diagnostic tests, including molecular beacons, as described above, which may detect a single polymorphism in a nucleic acid sequence; and conformational changes in antibodies, enzymes or receptors upon binding to their antigens, substrates or ligands, respectively, among others. These examples are merely illustrative of some of the utilities of hybrid molecules comprising a fluorophore and quencher, and is not intended to be limiting to any particular field of use of such molecules, such as the diagnostics, semiconductor, and other fields. Moreover, a hybrid molecule may have more than one fluorophore or more than one quencher. The fluorophore or the metal surface quencher of the invention may be affixed to a substrate such as a microarray chip, cuvette, plastic bead, flow-through tubing, or any other substrate in which or on which fluorescence can be detected by eye or by machine.

The present application claims priority under 35 U.S.C. 119(e) to provisional patent applications serial No. 60/228,728, filed Aug. 29, 2000, and 60/280,350, filed Mar. 30, 2001, both of which are incorporated by reference herein in their entireties.

The inventors have found by surprise the remarkable efficiency of quenching of fluorescence by metal surfaces, including particles and films, particularly for use in biological diagnostic systems such as molecular beacons, and such an increased efficiency and concomitant increase in the signal-to-noise ratio in diagnostic systems incorporating such fluorophore-metal surface quenchers offers a significant increase in sensitivity, detection limit, and utility of various molecular conformation detection systems in a wide variety of fields. As the ability to detect rare events or the presence of minute, amounts of a biomolecule is diagnostically useful but practically useless unless the signal of such an event can be identified above background (noise), the instant invention offers to vastly increase the detectability of such rare events or molecules and moreover reduce the difficulties and associated extreme methods that are presently required for such detection. Simplification and expansion of the variety of substances and events that can be detected and measured, and reduction in size of associated detectors, such as light emitters and detectors, is offered by the present invention. A non-limiting example of the value of ultrasensitive detection afforded by the methods of the present invention is in the early monitoring of potential rejection of transplanted organs by the expression of particular genes in minute quantities. Very early detection offers the ability for early intervention and what essentially is prophylaxis of the condition, rather than treatment. Generally, such sensitive methods allow for close monitoring of homeostasis and early warning of adverse changes which can be corrected before overt and irreversible pathology occurs. Detection of various analytes by the methods herein provides such opportunities. Moreover, and as will be elaborated on further below, the ability to identify the need for, and as a result, administer a potentially toxic therapeutic agent at a minimal dose corresponding to only that necessary to counteract an early adverse event provides a new therapeutic modality that may expand the utility of otherwise unusable pharmaceutical agents.

Moreover, the inventors herein have noted that quenching of radiation by metals comprising metal particles or metal surfaces, such as but not limited to the phenomenon of surface plasmon resonance, have heretofore been a hindrance in the sensitivity and specificity of devices utilizing surface plasmon resonance in a diagnostic utility. In contrast, the instant invention takes advantage of this otherwise adverse effect of metal surfaces to greatly increase signal-to-noise in systems intentionally employing metal surfaces as quenchers.

A bulk metal can either be transparent to a radiation, or reflect it completely, depending on the wavelength of the radiation. This property is common to any metal. At the surface of the bulk metal, a peculiar phenomenon occurs due to the change of environment of the atoms at the surface. These atoms are not surrounded by other atoms as they are in the bulk metal, and their electrons have special properties. These properties include surface plasmon resonance, which is a collective resonant wave of electrons that travel at the surface of the metal. When such wave exists, it can couple to the radiation that produced it, and absorption can be enhanced. The absorption is inversely proportional to the sixth power of the distance between the emitting particle and the surface of the metal.

As described herein, any detection system in which the interaction between a fluorophore and a quencher can be identified or quantitated is applicable to the present invention. By use of a metal surface, such as a metal nanoparticle or film, in place of another quencher such as an organic molecule, the significant increase in sensitivity resulting from an increased signal-to-noise ratio is provided.

The metal nanoparticles or metal clusters used herein as fluorescence quenchers are known in the art, for example, such as are described in U.S. Pat. No. 5,360,895, and as sold by Nanoprobes, Inc., (Yaphank, N.Y.). Such metal nanoparticles may be prepared from such metals as gold, silver, palladium, and other metals such as but not limited to those mentioned hereinabove. They may comprise from 3 up to about 10⁷ (10 million) metal atoms. For example, those described in U.S. Pat. No. 5,360,895 may be provided with clusters of 6, 8, 9, 11, 13, 55 or 67 gold atoms. They may also be provided with functionalized groups, such as a maleimide group, which can covalently bind to a sulfhydryl group, for instance, on a protein or on a derivatized nucleic acid, to covalently bind the metal nanoparticle to the biomolecule.

Preferably, the metal nanoparticles used in the invention herein is gold, and more preferably, the gold nanoparticles comprise more than 11 gold atoms, or have a diameter greater than 0.8 nm. Most preferably, the gold nanoparticles have about 67 gold atoms per nanoparticle, with a diameter of about 1.4 nm. For conjugation, the, nanoparticles are derivatized with N-propylmaleimide groups (such as Catalog No. 2020 or 2020A from Nanoprobes, Yaphank, N.Y.).

The methods of the invention as described herein for metal particles are equally applicable to metal films. A metal film can be smooth (as when gold is evaporated on a smooth surface), or it can be rough (as when gold colloids are adsorbed and partially melted on an evaporated gold surface). Both type of films can be used as fluorescence quenchers. Examples include those as may be provided on, for example, an indwelling probe on to which a fluorescently-conjugated biomolecule is attached. Interaction of the biomolecule with its binding partner results in a change in conformation of the biomolecule and attendant change in proximity between the fluorophore on the biomolecule and the metal film, resulting in a change in fluorescence quenching. Monitoring such changes by fluorescence provide a means for measuring the instantaneous levels of an analyte in a distal site, and/or continuous monitoring of analyte levels over time. Thus, highly sensitive indwelling biosensors based upon quenching of fluorescence by metal surfaces may be used in the medical diagnostics as well as other fields in which sensitive detection of particular analytes is desired. Such biosensors may read out the detected levels and/or be integrated into a reactive system which delivers a response to levels which change to certain predefined parameters. For example, delivery of insulin in response to elevated blood glucose levels in a glucose biosensor; delivery of anti-rejection agents at sub-toxic doses in response to very early indications of organ rejection; and other agents to maintain homeostasis within close tolerances before reaching the pathologic stage are embraced herein.

As mentioned above, the metal surface quenchers of the invention may be used in combination with any fluorophore whose light emission may be quenched by the metal nanoparticle. Non-limiting examples of classes of such fluorophores include luminescent semiconductors, such as quantum dots (see, for example, Chan et al., 1998, Science 281:2016-2018; Bruchez et al., 1998, Science 281:2013-2015; Murray et al., 1993, J., Amer. Chem. Soc. 115:8706-8715; Lakowicz et al., 1999, J. Phys. Chem. B 103:7613-7620); a fluorescent organic dye, a fluorescent protein or a fluorescent peptide.

Fluorescent organic dyes may be, for example, fluorescein, rhodamine, Texas Red, Cy5, acridine orange, 2,7-dichlorofluorescein, eosin, rose bengal, 1,2-dihydroxyanthraquinone, 1,4-dihydroxyanthraquinone, 1,8-dihydroxyanthraquinone, 1,3,8-trihydroxy-6-ethylanthraquinone, 1,2,5,8-tetrahydroxyanthraquinone, 1-aminonaphthalene, and 2-aminonaphthalene. Other organic dyes as well as derivatives of the above may be employed. Examples of fluorescent proteins and fluorescent peptides is described above.

As noted elsewhere herein, the increased sensitivity of detection of conformational changes afforded by the methods and uses of metal surfaces herein enables the simultaneous measurement of a large number of different fluorophores, each identifying or quantitating a different conformational change. For example, a mixture of molecular beacons comprising metal nanoparticles, each specific for a particular single-nucleotide polymorphism and each with a different fluorophore whose emission wavelength is capable of being detected in the presence of all of the other fluorophores, may be simultaneously used to identify one or more polymorphisms in a sample. Moreover, by use of fluorophores with the same excitation wavelength but different (but still metal nanoparticle quenchable) emissions, a single excitation wavelength may be used to simultaneously determine numerous conformational change events, whether molecular beacons, multiple antigens binding to antibodies, ligands to substrates, etc. Thus, a panel of diagnostic tests may be performed simultaneously and homogeneously by use of the teachings herein.

As noted in the examples below, a significant increase in signal-to-noise is afforded by the combination of a fluorophore and a metal particle quencher, more than double, preferably one order of magnitude, more preferably two orders of magnitude, and most preferably three orders of magnitude. This increase provides the increased sensitivity of conformational assays employing metal nanoparticles as quenchers, desirable for the detection of very low levels of analytes, particularly in the presence, of otherwise interfering substances.

In its broadest aspect, the fluorophore-quencher combination detects changes in proximity of the fluorophore to the quencher. In practice, such proximity changes are useful for detecting binding of one molecule to another, or measuring conformational changes in a single molecule. In the example of the binding of one molecule to another, a fluorophore may be provided on one member of a pair of molecules which have a tendency to associate (or dissociate) naturally, or have a tendency to associate (or dissociate) only after a particular event has occurred, such as phosphorylation. The other molecule may have a quencher provided at a site which becomes proximal to the fluorophore when the molecules associate. Thus, quenching of the signal occurs when the molecules are associated. This property may be used to identify events or substances which encourage or discourage association between the molecules. For example, a homogeneous fluorescent immunoassay may be prepared for detecting a protein analyte by providing an antibody to the analyte labeled with a fluorophore, and the analyte labeled with a quencher. In the absence of analyte in a sample, the fluorescent antibody and the analyte-quencher for an immunocomplex, quenching the fluorescence and producing no signal. In the presence of increasing amounts of the analyte, the binding of the antibody to the labeled analyte is proportionately reduced, reciprocally increasing fluorescence. The same assay may be performed to identify antibodies specific to the analyte in a biological sample, in the same fashion.

By way of non-limiting example, a highly sensitive homogeneous fluorescent immunoassay for West Nile Virus antigen in a whole blood sample of a human, bird or mosquito, may be prepared using a rhodamine-labeled antibody to the virus, and a gold nanoparticle-labeled viral coat protein. Inhibition of binding of the labeled reagents, i.e., the production of fluorescence, indicates presence of the virus in the sample. Molecular beacons to the viral nucleic acid may likewise be prepared for sensitive detection of virions in these samples. Likewise, IgE antibodies to cat allergens may be detected in an individual using labeled cat allergen and specific IgE antibody. The presence of cat-specific IgE in the sample will compete for binding to the labeled cat allergens and increase the fluorescent signal. In both of the foregoing assays, the derivatization of the binding pairs with the fluorophore and the metal nanoparticle will be done to ensure that the reagents are bound at the interacting portions of the molecules, such that on binding, the proximity of the fluorophore and quencher minimize fluorescence. One of skill in the art may readily determine such parameters. Such assays may also be adapted for use with metal films, as described above.

Conformational changes in a protein or other polymeric biomolecule may also be exploited for detecting the presence of certain analytes, or monitoring the participation of the biomolecule in a reaction. By labelling two portions of the molecule which conformationally interact with a fluorophore and a metal nanoparticle, events which alter the interaction between those portions of the molecule, can be detected. Simply, a highly sensitive temperature detector can be prepared using a temperature-sensitive biomolecule, in which the temperature proportionally alters the proximity of the fluorophore and quencher. A detector provides the light emission and detects, amplifies and reads out the emission. Such changes in proximity may be applied to sensors for ionic strength, using, for example, polymers which swell or undergo hysteresis relative to the salt concentration. In another embodiment, a protein which functions as a receptor for a particular ligand can be used to measure the presence and extent of the ligand by labelling the protein with a fluorophore and metal nanoparticle quencher at locations of the molecule which change proximity when the ligand binds.

The foregoing examples are merely illustrative of various uses of the combination of a highly sensitive fluorophore and quencher moiety, the latter provided by metal surfaces including particles and films, for biological analyte detection among other uses. One of skill in the art can readily devise other systems which similarly utilize the quenchers of the invention for other uses. Moreover, the skilled artisan also will readily devise means for providing the associated excitation source and emission detection and readout to translate the proximity change being sensitively detected and the readout of use to the particular application, whether level of a certain substance in a blood or urine sample, temperature, a cancer-related mutation in DNA, the presence of coliform bacteria in, drinking water, as mere examples of a large number of utilities of sensitive proximity detection assays. Of course, as mentioned above, a large number of such assays can be performed simultaneously in a single reaction if the fluorescence related to the presence or extent of each analyte can be discriminated. In addition, the increased sensitivity of the detection system may afford a corresponding decrease in the size and/or complexity of the associated instrumentation to emit, detect and compute the desired readout, providing the opportunity for miniaturization of analytical, and particularly medical, instrumentation, as well as perform such analyses in situ rather than requiring samples to be analyzed ex vivo.

In a further aspect, the invention is directed to a molecular beacon comprising a metal nanoparticle as quencher. The molecular beacon may be capable of detecting and discriminating any nucleotide sequence, and the fluorophore normally present thereon attached by routine methods. The metal nanoparticle may also be attached by routine methods, such as by using a polynucleotide sequence with a disulfide at one end, such as the 5′-end. The disulfide bond can be cleaved by use of a reducing agent and the sulflhydryl-reactive maleimide-derivatized metal nanoparticle covalently bound. Often, the quencher DABCYL is used in combination with a fluorophore for a molecular beacon; use of a metal nanoparticle in place of DABCYL provides a significant increase in signal-to-noise, as described elsewhere herein. Preferably, the metal nanoparticles used in the invention herein is gold, and more preferably, the gold nanoparticles comprising more than 11 gold atoms, or having a diameter greater than 0.8 nm. Most preferably, the gold nanoparticles having about 67 gold atoms per nanoparticle, with a diameter of about 1.4 nm.

The aforementioned methods are equally applicable to metal films, which may be derivatized for covalent binding to or interacting with various biomolecules in a similar fashion as the metal particles described above. Alternatively, a metal film or coating can be further coated with a layer of a polymer with functional groups and/or one or more that provide a hydrophobic, hydrophilic, or otherwise charged groups, or that subsequently may be derivatized to bear functional groups, for attachment to a fluorescently-labeled biomolecule. Furthermore, in a similar fashion to the above-described mixture of molecular beacons for a number of different analytes, each beacon with different fluorescences each of which may be separately discriminated, the same methods may be used with a metal film, in which a mixture of different analyte-sensitive biomolecules may be bound, each with a different fluorophore. Thus, a single sensor or bioprobe may be used to monitor levels or continuous changes in levels of a large number of analytes simultaneously, based on measuring fluorescence quenching at each particular wavelength. This method, as well as that of the metal particle-based mixtures, has the advantage of not requiring partitioning of the individual biomolecules among different areas, spaces, cells, or in any array pattern, as present biochip methods require. The discrimination of the extent of quenching of the individual biomolecules can be done in the presence of all of the other biomolecules, using, for example, a single optical fiber and associated fluorescence emission, detection, and electronics. The increase in signal-to-noise offered by the present methods offers a corresponding decrease in the complexity of the biological portion of the detection system, which when integrated with the present state-of-the-art with regard to microelectronics, provides monitoring possibilities previously unachievable.

Thus, the present invention is further directed to a composition comprising a metal surface as described hereinabove; a fluorpohore as described hereinabove, and a molecule whose conformational changes are desirably detected by the relative positions of the fluorpohore and metal surface associated with the molecule. Examples of each of these components have been described in detail herein, yet these are merely exemplary and any suitable member of each of the three components may be used without deviating from the teachings of the present invention.

The present invention may be better understood by reference to the following non-limiting Examples, which are provided as exemplary of the invention. The following examples are presented in order to more fully illustrate the preferred embodiments of the invention. They should in no way be construed, however, as limiting the broad scope of the invention.

### EXAMPLE 1

### Covalent Linkage of a Gold Nanoparticle and a Fluorophore to a Single Stranded DNA

Twenty-five-base long oligodeoxyribonucleotide 5′-S-S-GCGAGTTTTTTTTTTTTTTTCTCGC-NH₂-3′ (SEQ ID NO:1), that contained a disulfide group terminated by a trityl moiety at the 5 end and a primary amino group at the 3′ end was obtained from Midland Certified Reagent Company, TX. The disulfide group was covalently linked to the 5′ phosphate via a (CH₂)₆ spacer, and the primary amino group was linked to the 3′ hydroxyl via a (CH)₂)₇ spacer. Before coupling, the DNA product was first purified using reverse phase chromatography, to select only the oligodeoxyribonucleotides that have the complete sequence and the modifications including the disulfide group. Two consecutive reactions were then carried out. First, an amino-reactive dye, including fluorescein, rhodamine 6G and Texas Red (Molecular Probes, Eugene, Oreg.) or Cy5 (Amersham Pharmacia Biotech) were covalently linked to the 3′-amino group. Monomaleimido-Au particles (Nanoprobes, Yaphank, N.Y.) were then covalently linked to the 5′-sulthydryl group. The monomaleimido-Au particles are gold clusters, 1.4 nm in diameter, passivated with water soluble phosphine ligands (12 on average), and functionalized with one N-propylmaleimide.

In the first coupling reaction, 100 microliters of a solution containing 100 micromolar oligodeoxyribonucleotide dissolved in 0.1M sodium bicarbonate was reacted with 0.1 mg of a succinimidyl ester of the dye dissolved in 100 microliters of dimethyl sulfoxide. The reaction mixture was stirred at room temperature for two hours. The reaction product was first purified with a Sephadex column (NAP-5, Amersham Pharmacia Biotech) equilibrated with 10 ml of 0.1 M triethylammonium acetate (pH 6.5) and then fractionated on a C-18 reverse phase column (Biorad), with a linear elution gradient of 0 to 75% acetonitrile dissolved in 0.1 M triethylammonium acetate (pH 6.5), and run 45 min at a flow rate of 1 ml/min. The fraction that absorbs at 260 nm and at the dye maximum absorption was isolated. The fractions collected after the HPLC were partially dried in SpeedVac (Savant, Farmingdale, N.Y.) and mixed together. The mixture was purified again using a Sephadex column equilibrated with 0.1 M sodium bicarbonate (pH 8.3). The elution volume was concentrated with Ultrafree-0.5 centrifuge columns 5,000 MW cut-off (Millipore), so that the final oligonucleotide concentration is 15 micromolar.

Before coupling the gold, the disulfide was cleaved with DTT and the oligonucleotide was purified from excess DTT. Ten microliters of 1 M DTT was added to 25 microliters of oligonucleotide mixed with 75 microliters of sodium bicarbonate pH 8.3. After 1 hr incubation, the oligonucleotide solution was purified through a Sephadex column (NAP-5) equilibrated with water. Part of the elution product (37 pmol to 370 pmol of DNA, suspended in 180 microliters of water) was immediately reacted with 6 nmol of the monomaleimido-Au particles (Nanoprobes, Yaphank, N.Y.) in aqueous 20 mM NaH₂PO₄, 150 mM NaCl, 1 mM EDTA buffer, pH 6.5, containing 10% isopropanol at 4° C. for 24 h.

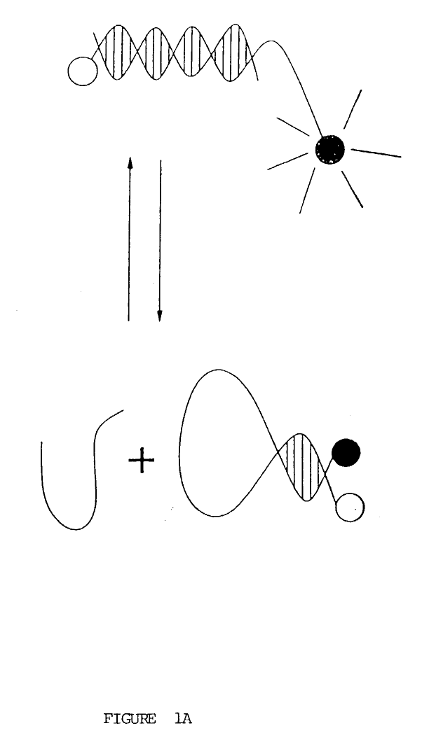

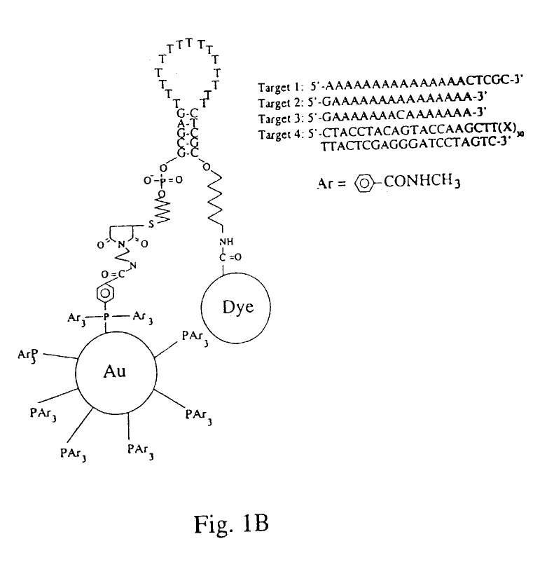

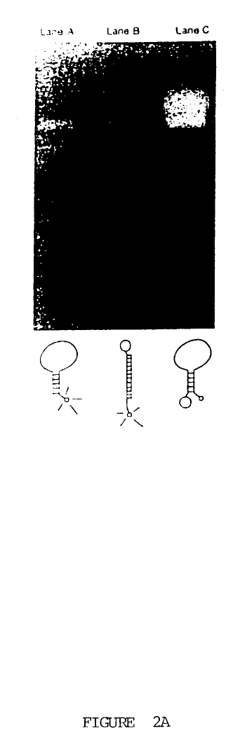

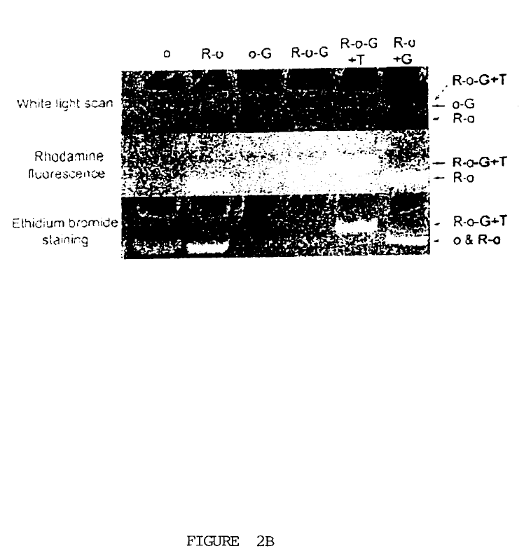

The structure of the product prepared as above is schematically illustrated in FIG. 1A, and in more detail in FIG. 1B. FIG. 1B also shows the targets used to quantify gold quenching (target 1; SEQ ID NO:2), and targets 2 (SEQ ID NO:3) and 3 (SEQ ID NO:4) for mismatch detection. The sequence was designed such that the hairpin structure is very stable at room temperature, but opens easily upon hybridization of the loop to its target. The reaction product was analyzed by gel electrophoresis (4% agarose gel without ethidium bromide, NuSieve, FMC Bioproducts, Rockland, Me., in 1×TBE buffer at 8V/cm) with direct visualization of the DNA through UV excitation of the dye. The dye-labelled nucleotides were visualized by UV excitation (black bands). FIG. 2, lane A, shows 15 pmole of fluorescein-DNA complex; lane B: a 10:1 mixture of target 1 with 18 pmole of dye-DNA-Au conjugate with 0.3 nmole of gold; and lane C: 180 pmole of dye-DNA-Au conjugate with 3 nmole of gold. The gold particles move in the electric field in opposite direction to the DNA: they are visible by eye and with UV excitation as a white smear above the loading well (lane C). The loading wells appear as sharp white bands in each lane.

The retention time of the dye-DNA conjugate is significantly shorter than the one of the gold-DNA-dye conjugate. Bands of gold clusters are visible to the naked eye and move in the opposite direction from the DNA, suggesting that they are positively charged. In the gel, a 10-fold dilution of gold-DNA-dye mixed with an excess of targets (FIG. 2A, lane B) yields a fluorescence greater than gold-DNA-dye alone (lane C).

FIG. 2B shows the results of 10% non-denaturing acrylamide gel electrophoresis performed in 1×TBE at 10V/cm. From left to right: lane 1: 125 pmol of non-conjugated oligonucleotide; lane 2: 125 pmol of rhodamine 6G-oligonucleotide conjugate; lane 3: 125 pmol of oligonucleotide reacted with 1.5 nmol of monomaleimido Nanogold; lane 4: 125 pmol of rhodamine 6G-oligonucleotide conjugate reacted with 1.5 nmol of monomaleimido Nanogold; lane 5: same as lane 4 with an excess of target 1; lane 6: 125 pmol rhodamine 6G-oligonucleotide conjugate without the 5′-disulfide group reacted with 1.5 nmol of monomaleimido Nanogold. Lanes 1 to 6 have been labeled with o, R-o, R-o-G, T+R-o-G, and R-o+G where “o” stands for “oligonucleotide”, “R” for “Rhodamine 6G”, “G” for “gold nanocluster”, and “T” for “target”, the dash indicating a covalent bond. The top photograph is a white light scan of the gel. Both the gold-oligonucleotide conjugates and the dye-oligonucleotide conjugates are visible with the naked eye. Under UV excitation (middle photograph), the dye-oligonucleotide complexes (lanes R-o, R-o-G+T and R-o+G) produce a strong fluorescence. The dye-oligonucleotide-gold conjugates (R-o-G) do not emit any visible light, but the same mixture in the excess of Target (lane R-o-G+T) yields a fluorescence similar in intensity to that of the dye-oligonucleotide conjugate (R-o). The bare oligonucleotides (lane o) appears with ethidium bromide staining (bottom photograph). Unconjugated gold nanoparticles do not penetrate the gel; they migrate in opposite direction from the DNA. Oligonucleotides missing the disulfide group do not interact with gold (lane R-o+G is similar to lane R-o).

### EXAMPLE 2

### Measure of the Gold Nanoparticle Quenching Efficiency

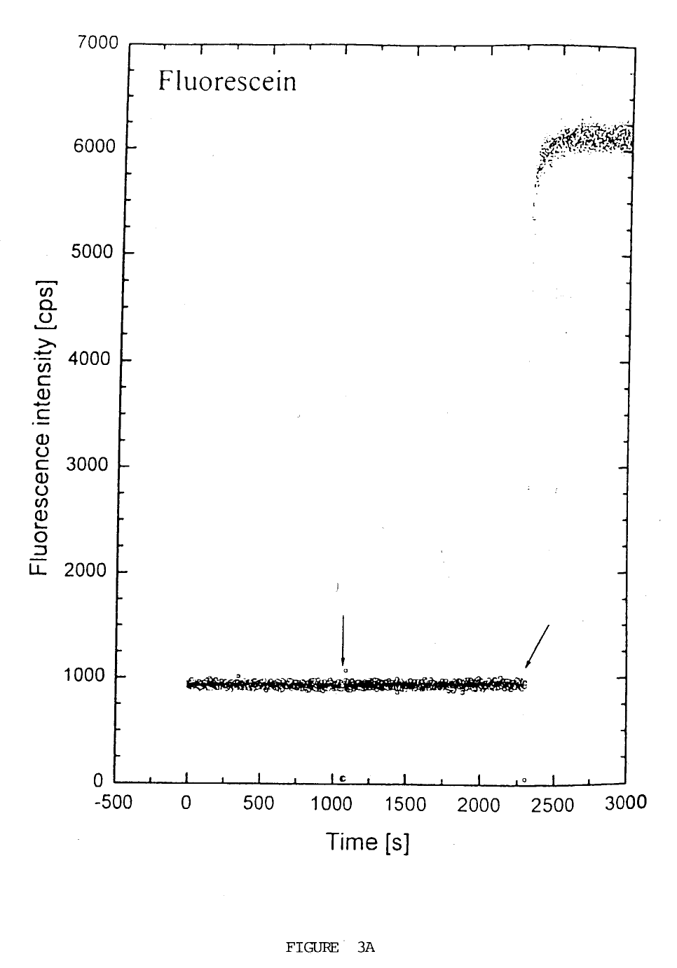

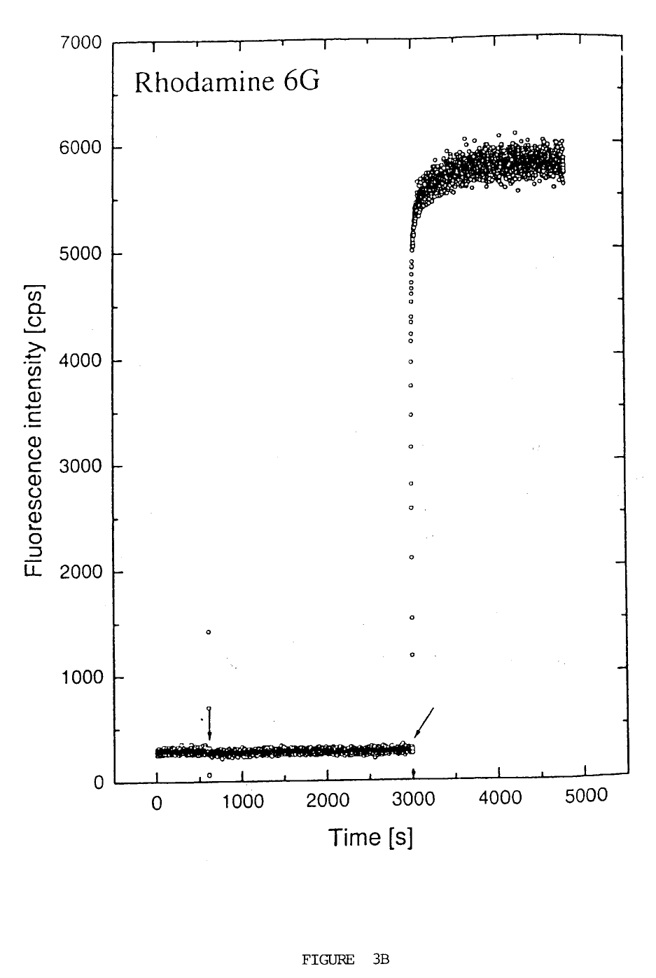

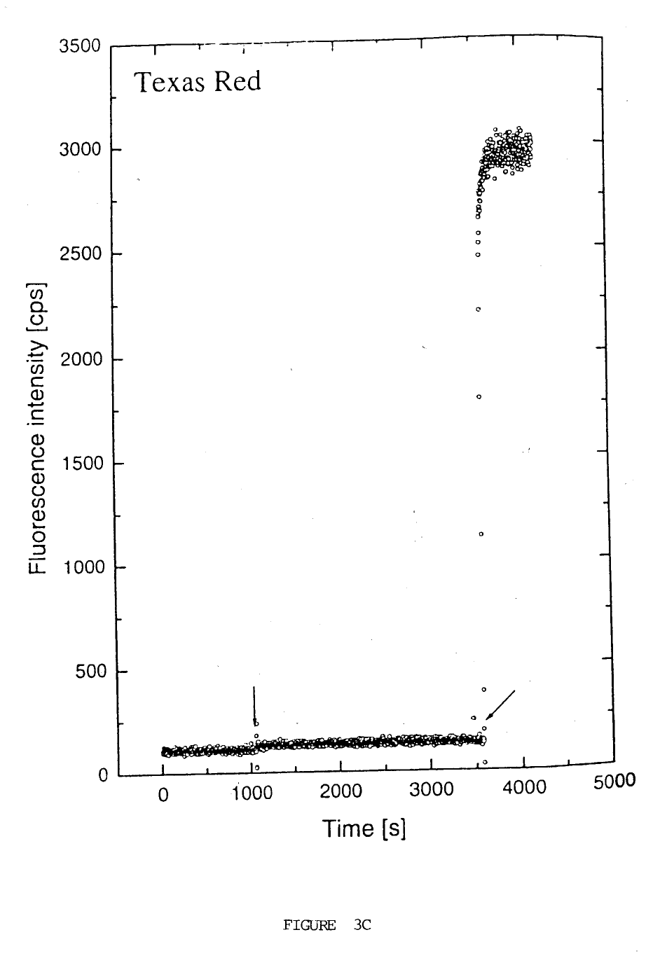

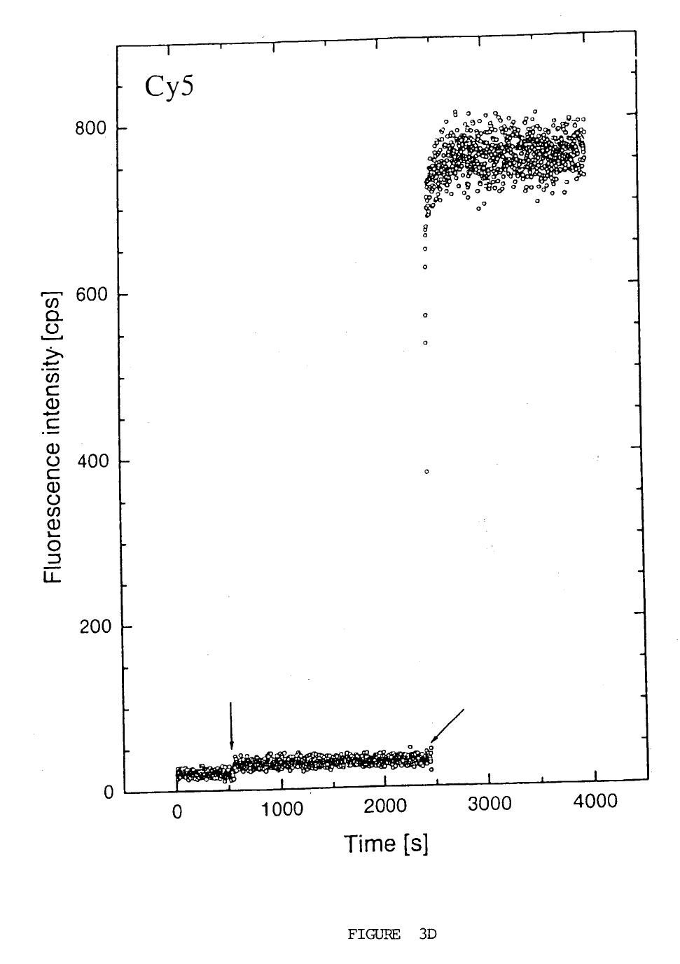

Fluorescent measurements were performed on a spectrofluorimeter (Photon Technology International, Monmouth Junction, N.J.), using a 10 mm path length quartz cuvette (NSG Precision Cells, Farmingdale, N.Y.) whose temperature was fixed to 20° C. with a circulating bath. The background fluorescence of 3 ml of 1M NaCl, 10 mM cacodylic acid, 0.5 mM EDTA (pH 7.0) was monitored for several minutes. Ten to 100 microliters of mixture of uncoupled gold and gold-DNA-dye conjugate was added to the hybridization buffer and fluorescence was measured. After confirming that there was no change of fluorescence with time, an excess of target oligodeoxyribonucleotide (5′-AAAAAAAAAAAAAAA-CTCGC-3′; SEQ ID NO:2) was added, and the level of fluorescence was recorded each second. This experiment was repeated with four constructs with four different dyes. For each dye, the excitation and the emission wavelengths of the fluorimeter were adjusted to match those given by the manufacturer of each dye: (494 nm; 520 nm) for Fluorescein (FIG. 3A), (524 nm; 557 nm) for Rhodamine 6G (FIG. 3B), (583 nm; 603 nm) for Texas Red (FIG. 3C), and (649 nm; 670 nm) for Cy5 (FIG. 3D).

The change of the level of fluorescence due to the gold particles was measured at the wavelengths used for each dye. Two nmole of pure Nanogold particles were suspended in the same conditions as the ones used for coupling to the oligodeoxyribonucleotide, and the change of fluorescence was measured as the mixture is added to the hybridization buffer.

As shown in FIG. 1A, single-stranded DNA conjugated at each extremity to a dye moiety and to a gold nanoparticle offers an elegant way to test the quenching efficiency of the gold nanoparticle. The DNA can adopt two conformations: a stem-loop structure where the fluorophore and the gold nanoparticle are held in close proximity (closed state; FIG. 2B), and a rod-like structure where they are far apart (open state). The ratio of the fluorescence of the open state to the close state, referred to as the signal to noise ratio (S/N) of the gold-quenched beacon, gives a direct measure of the quenching efficiency of the gold nanoparticle.

A high concentration of salt ensures that the single stranded DNA forms a hairpin. Then a mix of gold-oligonucleotide-dye conjugate and containing non-reacted gold is introduced in the cuvette. The fluorescence level changes slightly, depending on the quenching efficiency of the gold nanoparticle. In the last step, a single stranded oligonucleotide (the target) complementary to the loop and to half of the stem of the hairpin DNA (FIG. 1A) is mixed in excess. As the target hybridizes to the loop of the hairpin DNA, the fluorescence rises significantly.

When the mixture of gold and gold-oligonucleotide-dye conjugates is introduced in the cuvette, the intensity of the fluorescence is raised by the imperfect quenching of the dye, and decreased because of the absorption of part of the excitation light by the gold nanoparticles. These two competing phenomena may result in a total increase (FIG. 3C and 3D) or decrease of the fluorescence (FIG. 3A and 3B).

The offset due to the presence of the non-coupled gold particles is compensated by the measured fluorescence intensity absorbed by the gold nanoparticles in absence of oligonucleotides. The ratio of the fluorescence intensity measured in the cuvette before and after adding the target gives, after compensation for the background and the offset fluorescence, the signal to noise of the hybrid probe, as shown in the table below. For a given dye, the signal to noise of the hybrid probe depends strongly on the quality of the coupling of the gold nanoparticle to the dye-oligonucleotide conjugate. The samples with the best signal to noise ratio are presented for each dye in FIG. 3. The precise experimental measures are reported in the table, as well as the average signal to noise obtained for all the coupling performed for a given dye. Rhodamine 6G is the best quenched dye with an average signal to noise of 710+/−230. On 13 reactions studied, 4 produced a signal to noise greater than 1000, and the best sample had a signal to noise close to 3000.

Fluorescent intensities were measured in the spectrofluorometer using a 3 ml cuvette at 20° C., from (i) the buffer alone (1 M NaCl, 10 mM cacodylic acid, 0.5 mM EDTA, pH 7.3), (ii) the mixture of gold and gold-oligonucleotide-dye conjugates in the buffer, (iii) gold alone with concentration same as in (ii), (iv) same as (ii) after the addition of an excess of target. The signal to noise ratio (S/N) of the gold-oligonucleotide-dye is computed as (Iᵢᵥ-Iᵢᵢᵢ)/(Iᵢᵢ-Iᵢᵢᵢ). For each dye, the excitation and emission wavelengths of the fluorimeter were adjusted to the corresponding maximum of excitation and emission of the dye, as given by the manufacturer. In the case of fluorescein, only a lower bound for the signal to noise can be given. For rhodamine, the lower bound is 1300. The values reported here correspond to the samples presented in FIG. 3. The columns labeled N_{T} and <S/N> report the total number of dye-DNA-gold conjugation reactions for each dye (N_{T}), and the average signal to noise ratio (<S/N>) measured for all conjugates. The last column expresses the quenching efficiency (QE%) and the average quenching <QE> measured for all conjugates.

| | | | | gold + | | | | |
|---|---|---|---|---|---|---|---|---|
| | | gold + | | probe + | | | | |
| | Buffer | probe | gold | target | | | | QE % |
| Dye | (Iᵢ) | (Iᵢᵢ) | (Iᵢᵢᵢ) | (Iᵢᵥ) | S/N | N_{T} | <S/N> | <QE> |
| Fluorescein | 932 +/− 1 | 939 +/− 1 | 909 +/− 1 | 6121 +/− 2 | >500 | 4 | 170 +/− 94 | 99.42 +/− 0.02 |
| | | | | | | | | 98.68 +/− 0.47 |
| Rhodamine | 287.6 +/− 0.8 | 274.7 +/− 0.4 | 272.8 +/− 1.4 | 5794 +/− 3 | 2900 | 13 | 710 +/− 230 | 99.966 +/− 0.026 |
| 6G | | | | | | | | 99.45 +/− 0.54 |
| Texas Red | 108.8 +/− 04 | 135.2 +/− 0.3 | 93.5 +/− 1.0 | 2945 +/− 1 | 68 +/− 3 | 2 | 59 +/− 6 | 98.54 +/− 0.04 |
| | | | | | | | | 98.26 +/− 0.26 |
| Cy5 | 20.7 +/− 0.2 | 31.5 +/− 0.1 | 20.0 +/− 0.2 | 756 +/− 3 | 64 +/31 4 | 3 | 48 +/− 11 | 98.44 +/− 0.03 |
| | | | | | | | | 97.5 +/− 1.0 |

For each dye, the average signal to noise ratio when nanogold is used is greater or equal to the values obtained with regular molecular beacons.

The quenching efficiency of gold nanoparticles and DABCYL are compared in the following table. They were compared in high salt buffer (1 M NaCl, 10 mM cacodylic acid, 0.5 mM EDTA, pH 7.3) and in low salt buffer (90 mM KCl, 10 mM TRIS, pH 8.0) with the quenching efficiency of the gold quenched beacons described in the previous table. In both cases, only the best quenching efficiencies (QE) are reported. The standard errors are of the order of 0.05% for the DABCYL-quenched beacons.

| | QE (%) of DABCYL-quenched | | QE (%) of gold-quench- |
|---|---|---|---|
| | beacons | | ed beacons |
| Dye | High salt buffer | Low salt buffer | High or low salt buffers |
| Fluorescein | 98.59 | 96.55 | 99.42 +/− 0.02 |
| Rhodamine | 97.67 | 93.33 | 99.97 +/− 0.03 |
| 6G | | | |
| Texas red | 96.30 | 93.33 | 98.54 +/− 0.04 |
| Cy5 | 96.30 | 94.12 | 98.44 +/− 0.03 |

### EXAMPLE 3

### Single Mismatch Detection

For the mismatch detection, the hybridization buffer is composed of 90 mM KCl, 10 mM TRIS, pH8.0. One mismatch in a sequence of 16 bases was detected successively with two probes having the same oligodeoxyribonucleotide sequence, the same dye (rhodamine 6G) with either a Nanogold or a DABCYL as a quencher. 180 microliters of a solution containing 37 pmole of oligodeoxyribonucleotide and 4.8 nmole of monomaleimido-Au particles was split in three vials (1-3) containing each 60 microliters of the product. Vial 1 was mixed in 3 ml of the hybridization buffer and after few minutes with 100 microliters of 160 micromolar pool of oligonucleotides with a segment of 30 random bases flanked in between two 20 bases long segments. This pool of oligonucleotides acts as random targets competing with the perfect and the mismatched targets. The level of fluorescence was found to not change over 1 h. The same procedure was repeated with vial 2, but immediately after the addition of the random sequences, perfect targets (5′-GAAAAAAAAAAAAAAA-3′; SEQ ID NO:3) were added in steps. One min was allowed between each step, and the target concentration ranged from 67 pmolar to 13 micromolar. The same titration was repeated with vial 3 but with a target containing one mismatch (5′-GAAAAAAACAAAAAAA-3′; SEQ ID NO:4). Each target was suspended in the hybridization buffer so that the composition of the buffer does not change during the titration. A titration experiment lasted typically 30 min.

Similar titrations were done with the probe that have the DABCYL as quencher. The molecular beacon with a rhodamine 6G was synthesized by reacting a rhodamine 6G succinimidyl ester to the primary amine at the 5′-end of an oligonucleotide (5′-NH₂-GCG AGT TTT TTT TTT TTT TTC TCG C-3′-DABCYL; SEQ ID NO:1) that had a DABCYL attached at its 3′ end. After purification, the signal to noise of this molecular beacon is 35 in 1 M NaCl, 10 mM cacodylic acid, pH 7.0, it goes down to 8 in the buffer used for the .titration.

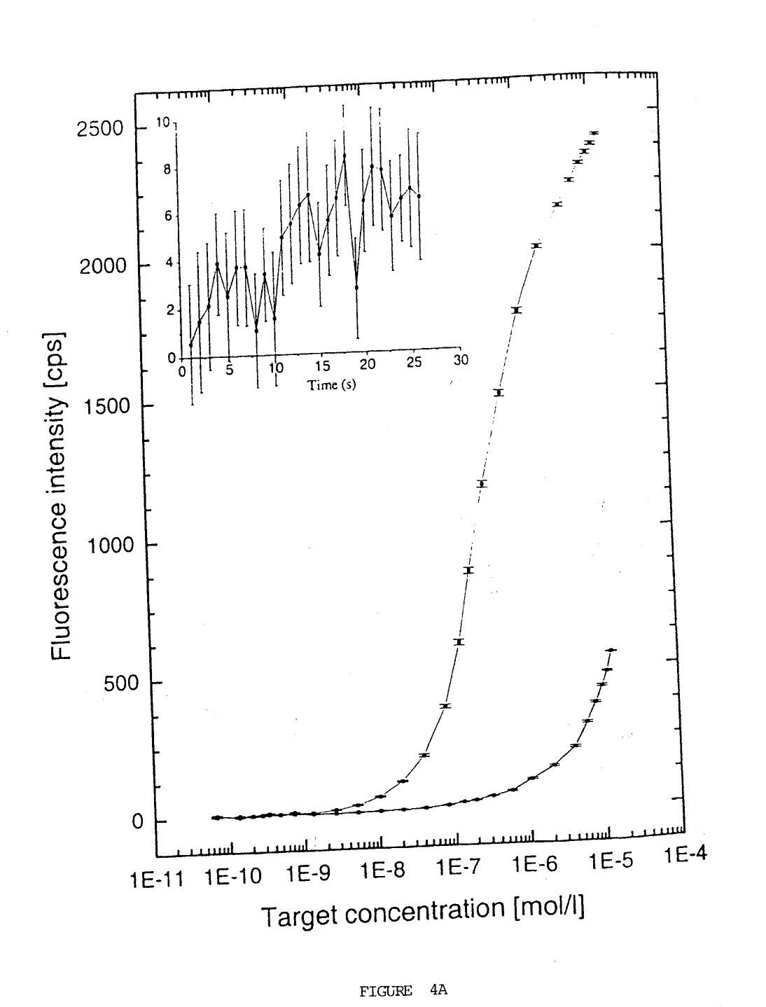

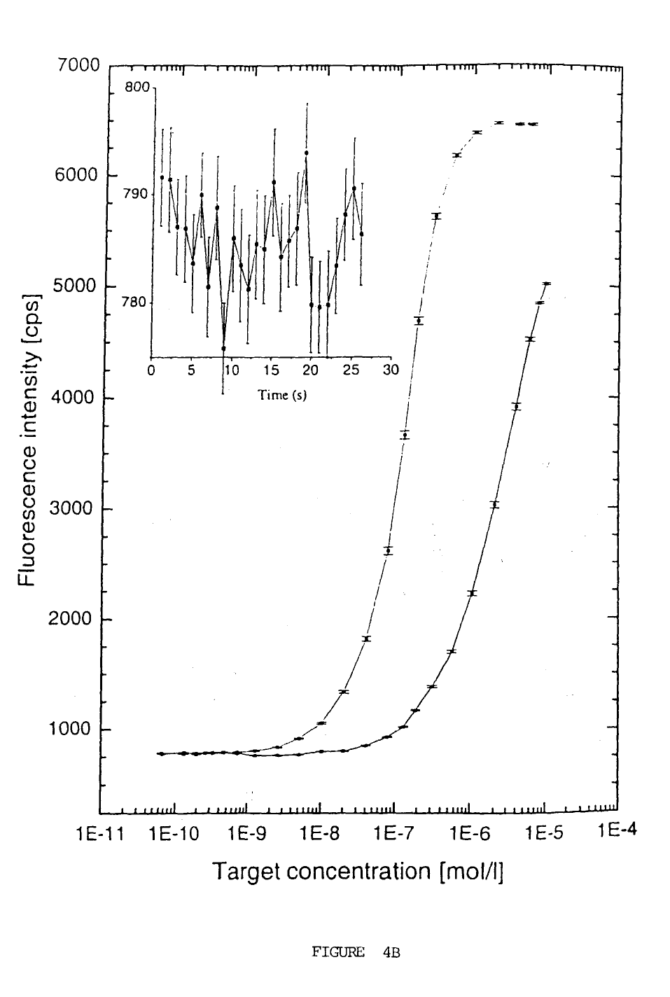

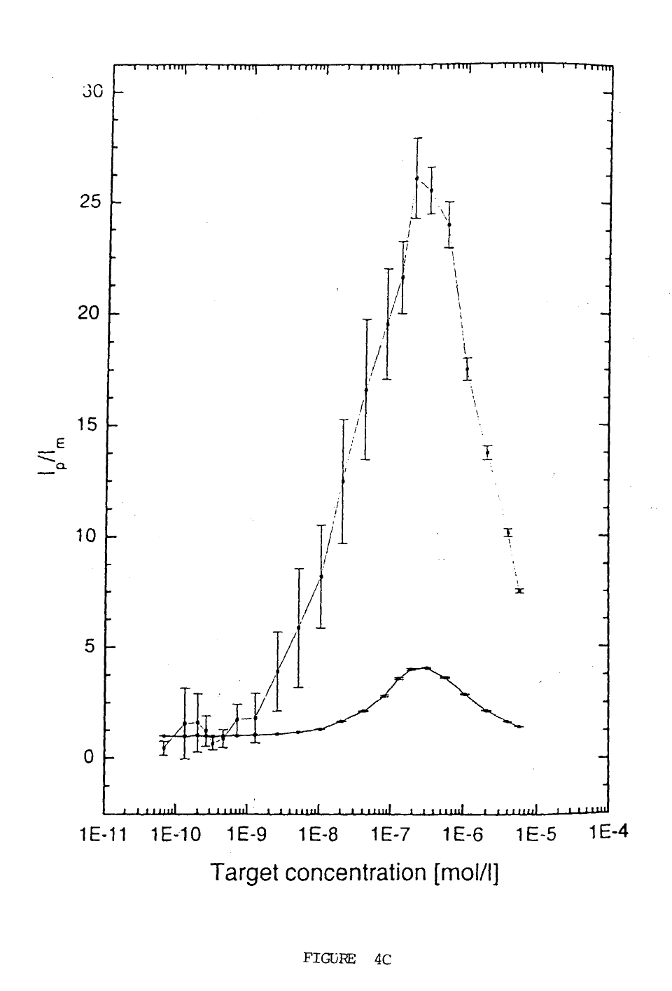

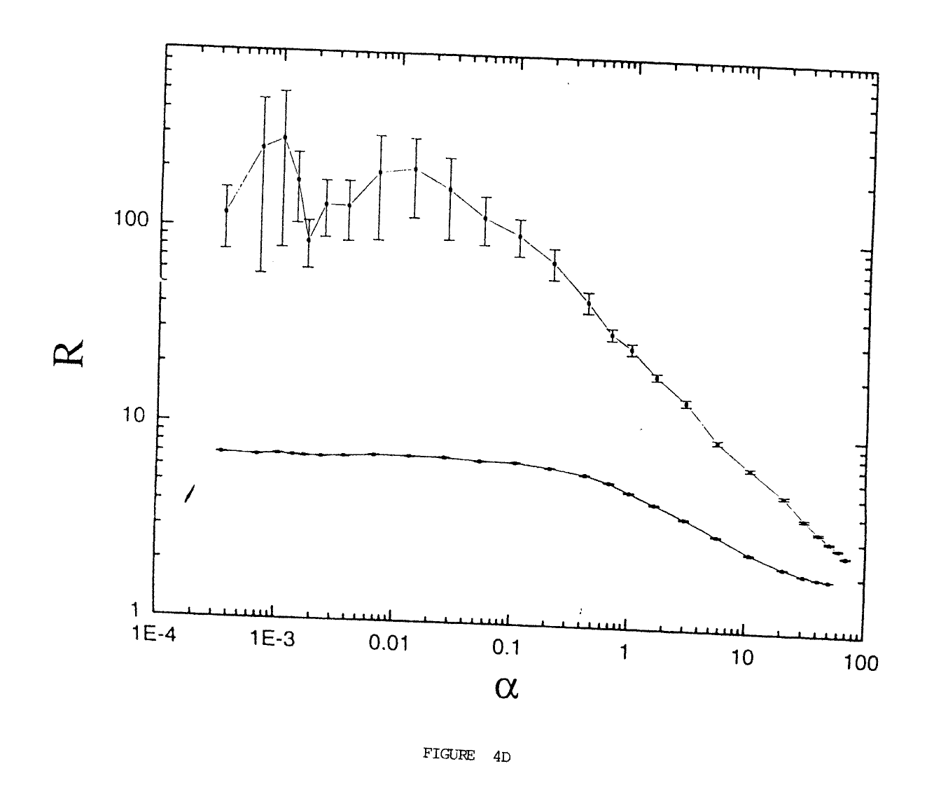

Two probes were used: a Rhodamine-DNA-gold conjugate (FIG. 4A) and a Rhodamine-DNA-DABCYL molecular beacon (FIG. 4B). The insets, show the evolution of fluorescence as a function of time when the probe is mixed with 5 micromolar of random targets (5′-CTACCTACAGTACCAAGCTT(X)₃₀TTACTCGAGGGAICCTAGTC-3′; X represents random bases differing from one DNA strand to another; target 4; SEQ ID NO:5). In both cases, the random targets do not induce any change of fluorescence of the probe during the time of titration. The hybridization is thus very specific to the matched of the mismatched targets.

With the two probes, it is possible to distinguish between the perfect target and the mismatch one. But the sensitivity to the mismatch detection are quite different. Let Iₚ(c) (resp. Iₘ(c)) be the absolute fluorescence intensity when the concentration c of perfect (resp. mismatch) targets are present in solution. The ratio Iₚ(c)Iₘ(c) (see FIG. 4C) gives the sensitivity to the perfect target compared to the mismatched target when they are present in equimolar concentration. For both probes, the best mismatch sensitivity is achieved around cₒ=0.2 micromolar, as shown FIG. 4C. At this concentration, with the dye-DNA-gold probe, Iₚ/Iₘ=25, whereas for the molecular beacon, Iₚ/Iₘ=4.

### EXAMPLE 4

### Competition Between Coexisting Targets

In the case of a mixed solution containing perfect targets, mismatched targets, and random targets, it is important to discriminate the perfect targets from the others (22). The resolution R of the DNA probe is the ratio between f(cₚ, cₘ, cₙ), the fluorescence of a solution of matched, mismatched and random targets at the respective concentration cₚ, cₘ, cₙ, and f(0, cₘ,cₙ), the fluorescence of a solution of mismatched and random targets only: R=f(cₚ, cₘ, cₙ)/f(0, cₘ,cₙ).

The strategy is to fix the concentration of perfect targets to C₀, the optimum concentration for mismatch detection, and to change the concentration of mismatched targets, cₘ. Since the random sequences do not bind to the probe (FIG. 4A and 4B), the optimal R is a function of one parameter: cₘ, R=f(c₀,cₘ)/f(0,cₘ)=(Iₚ(cₒ)+Iₘ(cₘ))/Iₘ(cₘ), and is conveniently presented in FIG. 4D as a function of α=c₀/cₘ. The threshold of resolution can be defined: the perfect target is said to be detected when R>3, which means that the fluorescence due to the perfect target is at least twice the one due to the mismatched one. In FIG. 3D, optimal R is plotted as a function of both for the gold-DNA-dye conjugate and for the molecular beacon. In the case of the hybrid probe, R>3 for α<50, thus if 1 out of 50 strands of DNA has the perfect sequence, it is detected. For the molecular beacon, R>3 for α<6, thus if 1 out of 6 strands of DNA have the perfect sequence, it is detected.

As shown in FIG. 4, evolution of the fluorescence of a solution containing A): 4.2 nM of gold-DNA-Rhodamine conjugate and 0.6 microM of gold, B): 10 nM of molecular beacon, as the target concentration varies from 67 pM to 13 microM. For both probes, the perfect target (target 2, solid line) produces a fastest and sharper increase of fluorescence than the target containing one mismatch (target 3, dotted line). The fluorescence due to the buffer and to gold (FIG. 4B) have been subtracted. This pool of random sequences was checked and does not affect the fluorescence level during titration time (dotted line).

In FIG. 4A, in the low salt buffer, in the absence of target, the absolute fluorescence due to the DABCYL-DNA-dye conjugate is much higher than background and its signal to noise ratio is close to 8 (it is 35 in higher salt buffer). In FIG. 4B, in the absence of target, the absolute fluorescence due to the gold-DNA-dye conjugate can not be distinguished from the background fluorescence, and its signal to noise ratio is close to 1000. In FIG. 4C, the ratio between the titration curve with the perfect target and the titration curve with the mismatched one. In FIG. 4D, the resolution of a match and a mismatch target, competing for hybridization. Molecular beacon (dashed line), Gold-DNA-dye conjugate (plain line). is the population ratio of match to mismatch targets. The concentration of perfect target is fixed to 0.2 micromolar.

### EXAMPLE 5

### Quenching of Hairpin-Shaped DNA on a Gold Surface

In this example, the hairpin-shaped DNA, 5′-Y-linker-GCG AGT TTT TTT TTT TTT TTCTCG-X-3′ (SEQ ID NO:6), has a fluorophore at one end and a functional group (either a thiol or a primary amine) at the other to facilitate binding to a gold surface, which in this case acts as the quencher. Y represents a disulfide or a primary amine; X can be any dye that can be linked to a primary amine, in one embodiment, fluorescein. The linker may be, by way of non-limiting example, either 1) C₆, C₉, C₁₈ or alkylthiol; 2) C═O-NH-(CH₂)₆-NH-C═O-(CH₂)₂-Y. The linker between the base and the dye is the same as that described in the foregoing examples. Use of a functional group to bind the DNA to the gold surface reduces non-specific binding. In addition to the use of a functional group, non-specific adsorption of biomolecules including DNA on metal surfaces may be reduced by other means known to one of skill in the art, such as but not limited to use of polymers to prevent the DNA from adsorbing onto the surface.

The gold surface or other metal film surface can be smooth, such as is prepared when gold is evaporated on a smooth surface, or it can be rough, such as is prepared by gold colloids adsorbed and partially melted on an evaporated gold surface.

Y can be directly attached on the gold in the case of a disulfide, or it can also be attached via functional polymers. The gold surface can be bare or treated with a polymer (such as dodecanethiol) as mentioned above to prevent non-specific adsorption of the DNA on the gold.

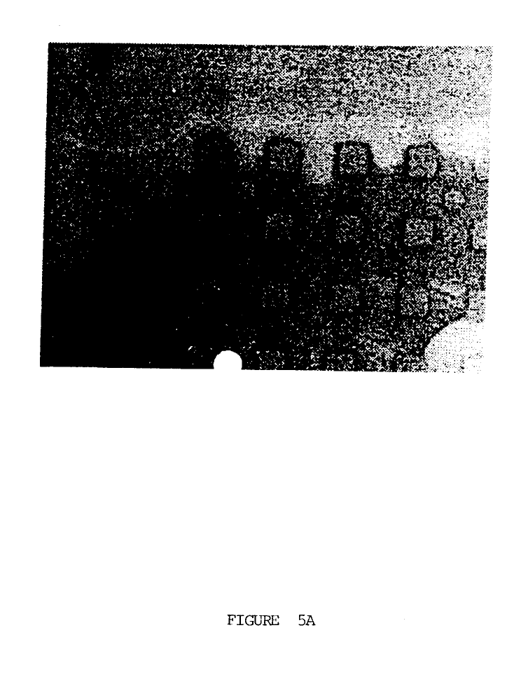

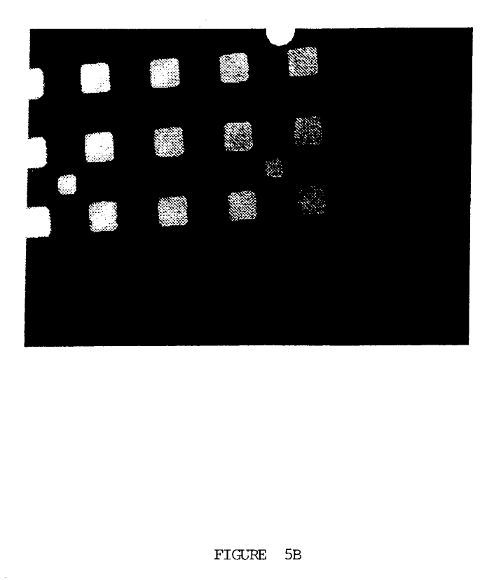

An array was prepared of chromium/gold squares (FIGS. 5A and B) and the aforementioned fluorescein-hairpin DNA coupled to the gold via thiol groups. The quenching efficiency of the gold surface was measured by comparing the fluorescence intensity when the DNA is opened (with fonmamide or with a complementary DNA sequence such as 5′-AAA AAA AAA AAA AAA CTC GC-3′ [SEQ ID NO:2]) or closed (in a salt solution, without target). A high degree of quenching was observed when the DNA is salt solution (FIG. 5A). On the contrary, in formamide (which opens up the hairpin DNA), some of the fluorescence is restored (FIG. 5B). The signal/noise ratio obtained is close to 8. Similar results have been obtained using DNA complementary sequences instead of formamide.

The present invention is not to be limited in scope by the specific embodiments describe herein. Indeed, various modifications of the invention in addition to those described herein will become apparent to those skilled in the art from the foregoing description and the accompanying figures. Such modifications are intended to fall within the scope of the appended claims.

It is further to be understood that all base sizes or amino acid sizes, and all molecular weight or molecular mass values, given for nucleic acids or polypeptides are approximate, and are provided for description.

Various publications are cited herein, the disclosures of which are incorporated by reference in their entireties.

What is claimed is:

1. A method for sensitively detecting proximity changes in a system that utilizes an interacting fluorophore and quencher, said method comprising utilizing as said quencher a metal surface.

2. The method of claim 1 wherein said metal surface is selected from the group consisting of a metal particle and a metal film.

3. The method of claim 1 or 2 wherein said metal is gold.

4. The method of claim 2 or 3 wherein said metal particle is a gold nanoparticle or a silver nanoparticle.

5. The method of claim 4 wherein said gold nanoparticle has a diameter greater than 0.8 nm.

6. The method of claim 5 wherein said gold nanoparticle has a diameter of about 1.4 nm.

7. The method of claim 4 wherein said gold nanoparticle has more than 11 gold atoms.

8. The method of claim 1 wherein said system comprises a hybrid molecule comprising a metal surface, a fluorophore, and a molecule whose conformation is desirably detected.

9. The method of any of claims 1 to 8 wherein said fluorophore is selected from the group consisting of a luminescent semiconductor, a fluorescent organic dye, a fluorescent protein or a fluorescent peptide.

10. The method of claim 9 wherein said luminescent semiconductor is a quantum dot.

11. The method of claim 9 wherein said fluorescent organic dye is selected from the group consisting of fluorescein, rhodamine, Texas Red, Cy5, acridine orange, 2,7-dichlorofluorescein, eosin, rose bengal, 1,2-dihydroxyanthraquinone, 1,4-dihydroxyanthraquinone, 1,8-dihydroxyanthraquinone, 1,3,8-trihydroxy-6-ethylanthraquinone, 1,2,5,8-tetrahydroxyanthraquinone, 1-aminonaphthalene, and 2-aminonaphthalene.

12. The method of claim 9 wherein said fluorescent protein is green fluorescent protein.

13. The method of an one of claims 1 to 12 wherein said increased sensitivity is an increased ratio of signal to noise.

14. The method of claim 13 wherein said ratio of signal to noise is increased over two fold.

15. The method of claim 14 wherein said ratio of signal to noise is increased over ten fold.

16. The method of claim 15 wherein said ratio of signal to noise is increased over a hundred fold.

17. The method of claim 16 wherein said ratio of signal to noise is increased over a thousand fold.

18. The method of claim 1 wherein said system measures a conformation change in at least one biomolecule.

19. The method of claim 18 wherein said at least one biomolecule is selected from the group consisting of a nucleic acid, a protein, a peptide, a glycoprotein, a glycolipid, and a polysaccharide.

20. The method of claim 19 wherein said nucleic acid is a molecular beacon.

21. The method of claim 19 wherein said protein is an antibody, a receptor, an enzyme or an enzyme substrate.

22. A composition comprising a molecular beacon wherein a quencher of said molecular beacon is a metal surface.

23. The composition of claim 22 wherein said metal surface is a metal nanoparticle or a metal film.

24. The composition of claim 23 wherein said metal nanoparticle is a gold nanoparticle.

25. The composition of claim 24 wherein said gold nanoparticle has a diameter greater than 0.8 nm.

26. The composition of claim 25 wherein said gold nanoparticle has a diameter of about 1.4 nm.

27. The composition of claim 24 wherein said gold nanoparticle has more than 11 gold atoms.

28. The composition of claim 22 wherein said metal surface is derivatized to covalently bind to form said molecular beacon.

29. The composition of claim 22 wherein said molecular beacon comprises a fluorophore selected from the group consisting of a luminescent semiconductor, a fluorescent organic dye, a fluorescent protein or a fluorescent peptide.

30. The composition of claim 29 wherein said luminescent semiconductor is a quantum dot.

31. The composition of claim 29 wherein said fluorescent organic dye is selected from the group consisting of fluorescein, rhodamine, Texas Red, Cy5, acridine orange, 2,7-dichlorofluorescein, eosin, rose bengal, 1,2-dihydroxyanthraquinone, 1,4-dihydroxyanthraquinone, 1,8-dihydroxyanthraquinone, 1,3,8-trihydroxy-6-ethylanthraquinone, 1,2,5,8-tetrahydroxyanthraquinone, 1-aminonaphthalene, and 2-aminonaphthalene.

32. The method of claim 29 wherein said fluorescent protein is green fluorescent protein.

33. A method for increasing the signal-to-noise ratio in a conformational-change-detectable hybrid biomolecule-fluorophore-quencher system in which the quencher is DABCYL, comprising substituting for DABCYL a metal surface.

34. The method of claim 33 wherein said metal surface is a metal particle or a metal film.

35. The method of claim 33 wherein said metal particle is selected from the group consisting of a gold nanoparticle and a silver nanoparticle.

36. The method of claim 35 wherein said metal nanoparticle is a gold nanoparticle.

37. The method of claim 36 wherein said gold nanoparticle has a diameter greater than 0.8 nm.

38. The method of claim 36 wherein said gold nanoparticle has a diameter of about 1.4 nm.

39. The method of claim 36 wherein said gold nanoparticle has more than 11 gold atoms.

40. The method of claim 33 wherein said metal surface is derivatized to covalently bind to form said hybrid molecule.

41. The method of claim 33 wherein said fluorophore is selected from the group consisting of a luminescent semiconductor, a fluorescent organic dye, a fluorescent protein or a fluorescent peptide.

42. The method of claim 41 wherein said luminescent semiconductor is a quantum dot.

43. The method of claim 41 wherein said fluorescent organic dye is selected from the group consisting of fluorescein, rhodamine, Texas Red, Cy5, acridine orange, 2,7-dichlorofluorescein, eosin, rose bengal, 1,2-dihydroxyanthraquinone, 1,4-dihydroxyanthraquinone, 1,8-dihydroxyanthraquinone, 1,3,8-trihydroxy-6-ethylanthraquinone, 1,2,5,8-tetrahydroxyanthraquinone, 1-aminonaphthalene, and 2-aminonaphthalene.

44. The method of claim 41 wherein said fluorescent protein is green fluorescent protein.

45. The method of claim 33 wherein said increased sensitivity is an increased ratio of signal to noise.

46. The method of claim 45 wherein said ratio of signal to noise is increased over two fold.

47. The method of claim 46 wherein said ratio of signal to noise is increased over ten fold.

48. The method of claim 47 wherein said ratio of signal to noise is increased over a hundred fold.

49. The method of claim 48 wherein said ratio of signal to noise is increased over a thousand fold.

50. The method of claim 33 wherein said system measures a conformation change in at least one biomolecule.

51. The method of claim 50 wherein said at least one biomolecule is selected from the group consisting of a nucleic acid, a protein and a polysaccharide.

52. The method of claim 51 wherein said nucleic acid is a molecular beacon.

53. The method of claim 51 wherein said protein is an antibody, a receptor, an enzyme or an enzyme substrate.

54. A composition comprising a covalent complex of a fluorophore, a metal surface quencher, and a molecule whose change in conformation is desirably detected, wherein a conformational change in said molecule is detectable by a change in fluorescence of said complex.

55. The method of any one of claims 1 to 53 wherein said metal surface is modified to provide a surface that is hydrophobic, hydrophilic, charged, functionalized, derivatizable, or any combination thereof.

56. The method of claim 54 wherein said modified surface is provided by attachment of a polymer or a ligand.
